(19) United States
(12) Patent Application Publication
(10) Pub. No.: US 2010/0063532 A1
(43) Pub. Date: Mar. 11, 2010

(54) Nasal Dilation Device

(76) Inventor: Corey C. Moore, Komoka (CA)

Correspondence Address: ADE & COMPANY INC., 2157 Henderson Highway, WINNIPEG, MB, R2G1P9, CA

(21) Appl. No.: 12/447,822

(22) PCT Filed: Oct. 15, 2007
(86) PCT No.: PCT/CA07/01808

§ 371 Date: Apr. 29, 2009

Related U.S. Application Data
(60) Provisional application No. 60/866,079, filed on Nov. 16, 2006.

(51) Int. Cl. A61M 29/00

(52) U.S. Cl. 606/199

(57) ABSTRACT

A nasal dilation device comprises a pair of dilating frames for insertion into respective nasal passages. An external abutment member is joined between the pair of dilating frames to abut an external nasal valve. Each dilating frame comprises a frame member wound in a generally helical pattern about a longitudinal axis of the dilating frame to form a plurality of windings spaced from one another in a direction of the longitudinal axis. By providing a frame formed of a helical member, a resulting open frame design of a dilating frame is provided which is easy to manufacture, of simple structure, with substantially no protruding ends or edges so that the device can be easily and safely inserted into a nasal passage without tearing or causing harm to the soft internal tissues thereof. Outward dilating pressure applied to the internal nasal passage is also distributed over a larger area for comfort.

This application claims priority to U.S. provisional application Ser. No. 60/866,079, filed Nov. 16, 2006.

### FIELD OF THE INVENTION

The present invention relates to a device for insertion into a nasal passage of a person for dilating the nasal passage to allow easier breathing by the person.

### BACKGROUND

Improving a person's ability to breathe is important where increased breathing is desired in sports, to reduce snoring, or for general health for example. It is known that breathing is restricted in many persons by a narrow nasal passage so that improved breathing can be accomplished by dilating or otherwise increasing the internal dimensions of the nasal passage of the person.

Various attempts have been made to improve breathing as disclosed in the following United States patents: U.S. Pat. No. 4,759,365 belonging to Askinazy; U.S. Pat. No. 6,978,781 belonging to Jordan; U.S. Pat. No. 6,270,512 belonging to Rittmann; U.S. Pat. No. 6,004,342 belonging to Filis; U.S. Pat. No. 3,710,799 belonging to Caballero; U.S. Pat. No. 5,895,409 belonging to Mehdizadeh; U.S. Pat. No. 1,672,591 belonging to Wells; U.S. Pat. No. 1,256,188 belonging to Wilson; and U.S. Pat. No. 7,105,008 belonging to Maryanka.

Prior art designs of nasal dilating devices are generally cumbersome so that the device themselves partially restrict air passage through the nasal passage. While some designs do have an open frame, in general known open frame designs either include edges which may be caught on sensitive internal tissues within the nasal passage during insertion or removal, or are difficult to manufacture due to the complex configuration thereof, or the devices do not include a sufficient number of frame members in an open frame design to evenly balance the outward pressure for dilating the nasal passage against the soft internal tissues of the nasal cavity.

### SUMMARY OF THE INVENTION

According to one aspect of the invention there is provided a nasal dilation device comprising:

at least one dilating frame arranged to be inserted into a nasal passage of a person to dilate the nasal passage;

said at least one dilating frame comprising a frame member wound in a generally helical pattern about a longitudinal axis of the dilating frame to form a plurality of windings spaced from one another in a direction of the longitudinal axis.

By providing a frame formed of a helical member, a resulting open frame design of a dilating frame is provided which is easy to manufacture, of simple structure, with substantially no protruding ends or edges so that the device can be easily and safely inserted into a nasal passage without tearing or causing harm to the soft internal tissues thereof. By providing lots of windings in the helical pattern of the frame, outward dilating pressure applied to the internal nasal passage is distributed over a larger area to further reduce the possibility of damaging or irritating the soft internal tissues of the nasal cavity.

The frame member is preferably formed of resilient material.

When there is provided an external abutment member arranged to abut an external nasal valve of the person, the abutment member is preferably spaced from said at least one dilating frame by a connecting portion extending therebetween generally in the direction of the longitudinal axis.

The connecting portion preferably has a length which is near a length of said at least one dilating frame in the direction of the longitudinal axis.

Said at least one dilating frame is preferably reduced in diameter adjacent an inner end thereof opposite the abutment member and may be resiliently compressible in a radial direction.

The external abutment member and connection of the connecting portion to said at least one dilating frame may be diametrically opposite one another in relation to the longitudinal axis.

When two dilating frames are provided, they are preferably commonly connected to the external abutment member by respective connecting portions connected to the dilating frames respectively at opposing external sides of the frames which are farthest from one another.

The dilating frames and the abutment member are preferably formed integrally with one another of a single continuous frame member of resilient material.

In some embodiments, at least the external abutment member is formed of a transparent material. Alternatively, the entire device may be formed of a transparent material.

The dilating frames are preferably arranged to be located by the abutment member and the connecting portions at any internal nasal valve of the person.

The external abutment member may generally lie within a vertical plane which is oriented parallel to and spaced laterally outward from the longitudinal axes of the dilating frames for optimally locating the dilating frames when the abutment member sits midcolumella on the user.

There may be provided a resilient coating on the frame member which is softer than the frame member.

The external abutment member may have a skin tone colouring either due to the colouring of the material forming the abutment member or by a coating.

When there is provided a coating on the frame member, the coating may comprise a medicinal compound arranged to be delivered to a wearer of the device.

According to a second aspect of the present invention there is provided a nasal dilation device comprising:

a pair of dilating frames, each arranged to be inserted into a nasal passage of a person to dilate the nasal passage; and

an external abutment member joined between the pair of dilating frames and arranged to abut an external nasal valve of the person when the dilating frames are inserted into the nasal passages of the person;

the external abutment member being connected to the dilating frames respectively at opposing external sides of the frames which are farthest from one another.

When connecting two dilating frames together to form the nasal dilation device, connecting the abutment member at respective outer sides of the dilating frames at locations farthest from one another results in an abutment member which does not cause any discomforting pinching at the external nasal valve or at the outlet of the nostrils.

According to a further aspect of the present invention there is provided a nasal dilation device comprising:

a pair of dilating frames, each arranged to be inserted into a nasal passage of a person to dilate the nasal passage; and

an external abutment member joined between the pair of dilating frames and arranged to abut an external nasal valve of the person when the dilating frames are inserted into the nasal passages of the person;

at least the external abutment member being formed of transparent material.

By further providing a connecting portion or abutment portion which is clear, all external portions of the nasal dilation device are transparent so that the device can be readily worn in public without drawing undesirable attention to the user.

Some embodiments of the invention will now be described in conjunction with the accompanying drawings in which:

### BRIEF DESCRIPTION OF THE DRAWINGS

FIG. 1 is front elevational view of the nasal dilation device.

FIG. 2 is a side elevational view of the device.

FIG. 3 is a bottom plan view of the device.

FIG. 4 is a top plan view of the nasal dilation device.

FIG. 5 is a partially sectional front elevation view of a nasal passage within which the nasal dilation device is shown schematically inserted.

FIG. 6 is a sectional view of the frame member including a coating thereon.

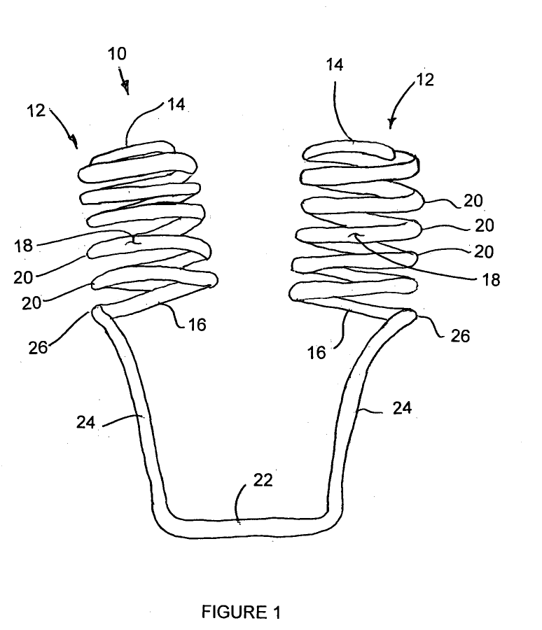

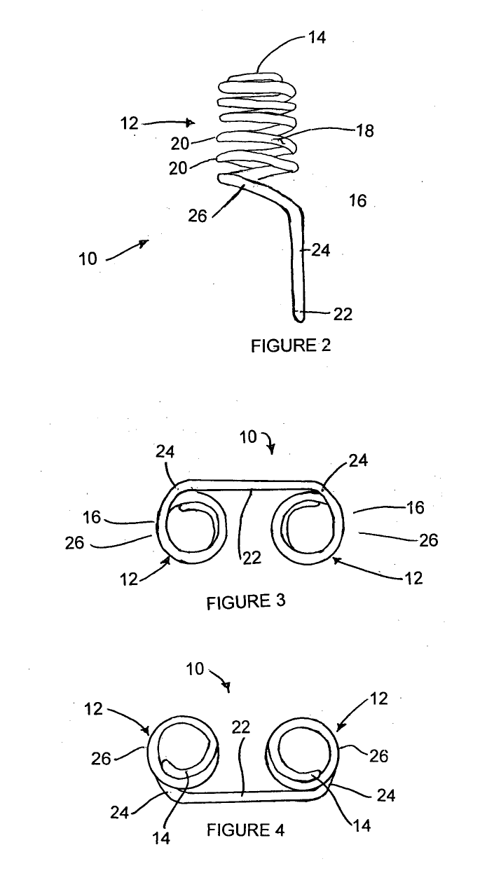

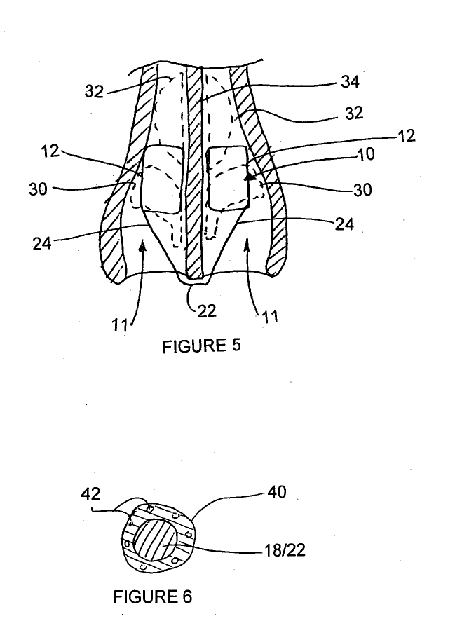

In the drawings like characters of reference indicate corresponding parts in the different figures.

### DETAILED DESCRIPTION

Referring to the accompanying figures there is illustrated a nasal dilation device which is generally indicated by reference numeral 10. The device 10 is particularly suited and arranged to be inserted into the nasal passages of a person for increasing the internal dimensions thereof by providing an outward pressure at the internal nasal valve located up inside the anterior roof of the nasal passages 11.

The device generally comprises a pair of dilating frames 12. Each of the frames 12 is arranged to be inserted into a respective one of the nostrils or nasal passages 12 of the person for dilating the respective nasal passage. Each frame 12 comprises an open frame forming a generally cylindrical outer shape about a longitudinal axis extending between an internal top end 14 which is open and an external bottom end 16 which is also open.

Each frame 12 is formed of a single frame member 18 which is wound in a helical pattern about the longitudinal axis of the frame between the opposed ends 14 and 16. Each frame member 18 is wound to define a plurality of individual windings 20, each comprising a portion of the frame member forming one complete revolution or circumference about the longitudinal axis of the frame.

Dimensions of the frame member 18 in cross section are arranged to be smaller than the space between windings to define the open frame by spacing the windings relative to one another in the direction of the longitudinal axis. The diameter of the last winding most adjacent to the internal top end 14 is reduced so that the frame 12 tapers inwardly towards the internal top end 14.

A free end portion of the frame member 18 projects slightly radially inwardly towards the longitudinal axis of the frame so as to terminate at position radially inward in relation to the outer circumference of the frame so that the edge of the free end portion is protected from contacting the internal tissues of the nasal passage as the frame is inserted or withdrawn from the nasal passage.

The external bottom ends 16 of the frames, which are opposite the internal top ends 14 of reduced diameter, are joined by external abutment member 22. Each frame 12 includes an integral connecting portion 24 which joins the frame respectively to the abutment member 22. The connecting portions 24 extend generally in the direction of the longitudinal axis of the respective frames from the external bottom end 16 of the frames to the abutment member 22 a length which is substantially equal or near to the length of the frames 12 in the direction of the longitudinal axis. The external abutment member 22 is thus arranged to abut the external nasal valve at the exterior of the nose between the nasal passages or nostrils while properly positioning the dilating frames 12 at the internal nasal valve.

Each connecting portion 24 joins the respective dilating frame 12 at the external end 16 at an external or outer side 26 of the frame farthest from the opposing frame 12 so that as the connecting portions 24 extend from the frames to the abutment member, the connecting portions also taper inwardly and extend towards one another as they approach the external bottom end at the abutment member.

The abutment member 22 spans a lateral distance between the respective longitudinal axes of the dilating frames 12 in a generally radial direction in relation to the two longitudinal axes. In relation to each frame, the abutment member extends generally radially outward therefrom beyond the cylindrical circumference defined by the frame in a direction which is diametrically opposite from connection of the respective connecting portion 24 to the frame 12.

The abutment member 22 generally lies in a vertical plane which is parallel to and spaced laterally outward equidistantly from the longitudinal axes of the dilating frames 12. The dilating frames are thus laterally offset from said vertical plane within which the abutment member 22 lies so that the abutment member is arranged to sit in the middle of the columella (outside portion between the two nostrils) for optimal camouflage of the device and comfort for the user in use. Offsetting the dilating frames 12 from the vertical plane of the abutment member 22 arranges the dilating frames 12 to be properly located in the internal nasal valve when the abutment member 22 sits midcolumella.

The two dilating frames 12, the connecting portions 24 formed integrally therewith and the abutment member 22 joined between the connecting portions 24 are all formed of a single continuous member which as been deformed to assume the shape as shown and described herein. The device is formed of a suitable material which has sufficient stiffness to retain its shape and provide structural support to expand the dimensions of the nasal passage when inserted therein by providing outward external pressure to internal nasal valve. The material forming the device however remains somewhat flexible and can be resiliently deformed for improving fit and comfort within the nasal passages of the person. By providing flexible connecting portions 24 or a flexible abutment member 22, spacing between the two dilating frames 12 can be adjusted for accommodating different dimensions of nasal passages of users.

In some embodiments, the frame member 18 forming the dilating frames 12 is also resiliently deformable so that the dilating frames are resiliently compressible in a radial direction to vary the overall diameter thereof and thus provide some degree of adjustability for accommodating different dimensions of nasal passages of different users.

The device 10 is also available in different sizes in which the diameter of the dilating frames 12 and the length of the connecting portions 24 between the frames and the abutment member are proportionally varied between small and larger size models.

As shown in FIG. 6, in some embodiments, the frame members 18 forming the dilating frames or the abutment member 22 include a coating 40 thereon. The coating can be applied by dipping in a silicone solution, for example, to produce a silicone coating thereon. A silicone coating which is softer than the material of the frame members increases the comfort for the wearer of the device.

When a coating 40 is provided, in some embodiments the coating further comprises a medicinal compounded 42 impregnated into the coating and arranged for delivery to the wearer. Desirable medicinal compounds would include nasal decongestants and the like.

In preferred embodiments the abutment member 22, or the entire device 10, are formed of a transparent material so that the device can be worn in public without drawing considerable attention.

Alternatively, when a coating is provided as shown in FIG. 6, the coating may have a skin tone colouring on at least the abutment portion so that the abutment portion 22 is disguised and blends in with the skin of the wearer in use.

In use, when it is desired to improve a person's ability to breathe through their nasal passage, the device 10 as described herein is inserted by inserting each of the dilating frames 12 into a respective nasal passage 11 of the user until the abutment member abuts the external nasal valve or external portion of the nose between the two nostrils. The user can then breathe in a normal manner while noticing a reduced restriction of airflow through the nasal passages. The device can be removed at any time by simply withdrawing the dilating frames 12 through the external nasal valve.

In further embodiments, windings of the frame member adjacent the external end where the connecting portions 24 are mounted may also be reduced in dimension to reduce the diameter or circumference thereof for improved comfort. The dilating frames in this instance generally have the shape of a barrel or beehive.

The internal nasal valve as defined herein is an area or cleft in the anterior roof of the nose that is bordered by the lower lateral cartilage 30, the upper lateral cartilage 32 and the nasal septum 34 in each nasal passage. An internal nasal valve angle less than 10° to 15° in the nose can cause nasal obstruction. As airflow is proportional to the radius of the nasal passages raised to the fourth power, a small change in the angle of the valve will have a large effect on the airflow and the resistance of the nasal cavity. Furthermore when air flows through a narrow space, such as the internal nasal valve, flow not only speeds up, it also creates an inward pressure which draws in potentially weakened nasal cartilages in worsening nasal breathing. By using a helically wound frame member to form the dilating frames, air flow is permitted through the open frame design in many directions while decreasing potential pressure points. The design also acts to stent or open up the external nasal valve or nostril. The helical pattern of the frame member forming the dilating frames is particularly advantageous due to its open design which allows air to pass freely between the helical coils or windings in many different directions, that is from underneath, over top and through it, etc., rather than flowing through in a unidirectional manner. This stimulates natural air flow through the nose much better.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A nasal dilation device comprising:
at least one dilating frame arranged to be inserted into a nasal passage of a person to dilate the nasal passage;
said at least one dilating frame comprising a frame member wound in a generally helical pattern about a longitudinal axis of the dilating frame to form a plurality of windings spaced from one another in a direction of the longitudinal axis.

2. The device according to claim 1 wherein the frame member is formed of resilient material.

3. The device according to claim 1 wherein said at least one dilating frame is resiliently compressible in a radial direction.

4. (canceled)

5. (canceled)

6. The device according to claim 1 wherein there is provided an external abutment member arranged to abut an external nasal valve of the person, the abutment member being spaced from said at least one dilating frame by a connecting portion extending therebetween generally in the direction of the longitudinal axis.

7. The device according to claim 6 wherein the connecting portion has a length which is near a length of said at least one dilating frame in the direction of the longitudinal axis.

8. (canceled)

9. The device according to claim 6 wherein the external abutment member and connection of the connecting portion to said at least one dilating frame are diametrically opposite one another in relation to the longitudinal axis.

10. (canceled)

11. The device according to claim 1 wherein said at least one dilating frame comprises a pair of dilating frames connected to one another by an external abutment member arranged to abut an external nasal valve of the person when the dilating frames are inserted into the nasal passages of the person.

12. (canceled)

13. The device according to claim 11 wherein the external abutment member is connected to the dilating frames respectively at opposing external sides of the frames which are farthest from one another.

14. The device according to according to claim 11 wherein the external abutment member is formed of resilient material.

15. The device according to claim 11 wherein at least the external abutment member is formed of a transparent material.

16. The device according to claim 11 wherein the external abutment member generally lies within a vertical plane which is oriented parallel to and spaced laterally outward from the longitudinal axes of the dilating frames.

17. (canceled)

18. (canceled)

19. (canceled)

20. The device according to claim 1 wherein there is provided a resilient coating on the frame member which is softer than the frame member.

21. The device according to claim 1 wherein there is provided an external abutment member arranged to abut an external nasal valve of the person, at least the external abutment member having a skin tone colouring.

22. The device according to claim 1 wherein there is provided a coating on the frame member comprising a medicinal compound arranged to be delivered to a wearer of the device.

23. A nasal dilation device comprising:
a pair of dilating frames, each arranged to be inserted into a nasal passage of a person to dilate the nasal passage; and
an external abutment member joined between the pair of dilating frames and arranged to abut an external nasal valve of the person when the dilating frames are inserted into the nasal passages of the person;
the external abutment member being connected to the dilating frames respectively at opposing external sides of the frames which are farthest from one another.

24. (canceled)

25. (canceled)

26. (canceled)

27. (canceled)

28. The device according to claim 23 wherein the abutment member is spaced from the dilating frames by a connecting portion extending therebetween generally in the direction of the longitudinal axis.

29. (canceled)

30. (canceled)

31. The device according to claim 28 wherein the external abutment member and connection of the connecting portion to the dilating frames are diametrically opposite one another in relation to the longitudinal axis.

32. (canceled)

33. (canceled)

34. (canceled)

35. (canceled)

36. The device according to claim 23 wherein the external abutment member generally lies within a vertical plane which is oriented parallel to and spaced laterally outward from the longitudinal axes of the dilating frames.

37. (canceled)

38. (canceled)

39. (canceled)

40. (canceled)

41. The device according to claim 23 wherein at least the external abutment member has a skin tone colouring.

42. (canceled)

43. A nasal dilation device comprising:
a pair of dilating frames, each arranged to be inserted into a nasal passage of a person to dilate the nasal passage; and
an external abutment member joined between the pair of dilating frames and arranged to abut an external nasal valve of the person when the dilating frames are inserted into the nasal passages of the person;
at least the external abutment member being formed of transparent material.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)
